(19) United States
(12) Patent Application Publication
(10) Pub. No.: US 2007/0286828 A1
(43) Pub. Date: Dec. 13, 2007

(54) Varnish composition based on a solvent exclusively of vegetable origin

(75) Inventors: Alain Deswartvaegher, Bergerac (FR); Bernard Forestier, Saint Nexans (FR); Sophie Miard, Saint Germain Et Mons (FR); Jean-Pierre Senet, Buthiers (FR); Sophie Thiebaud-Roux, L'Union (FR); Daniela Cristea, Toulouse (FR); Pascale De Caro, Toulouse (FR); Geraldine Giacinti, Fonsorbes (FR); Mathieu Bandres, Toulouse (FR)

Correspondence Address: HARNESS, DICKEY & PIERCE, P.L.C., P.O. BOX 828, BLOOMFIELD HILLS, MI, 48303, US

(73) Assignee: Durlin France, Bergerac (FR)

(21) Appl. No.: 11/805,553

(22) Filed: May 23, 2007

Related U.S. Application Data
(63) Continuation of application No. PCT/EP05/56098, filed on Nov. 21, 2005.

(30) Foreign Application Priority Data
Nov. 23, 2004 (FR) 0412409

(51) Int. Cl. A61K 8/34; A61K 8/37

(52) U.S. Cl. 424/61

(57) ABSTRACT

A varnish composition for cosmetic or pharmaceutical use, comprising at least one solvent of plant origin.

### CROSS-REFERENCE TO RELATED APPLICATIONS

This application is a continuation of PCT/EP2005/056098, filed Nov. 21, 2005, which claims priority to French Application No. 04/12409, filed Nov. 23, 2004. Both of these applications are incorporated by reference herein.

### BACKGROUND AND SUMMARY

The present invention relates to a varnish composition for cosmetic or pharmaceutical use, produced using solvents of natural origin. These solvents are acetates, carbonates or ethers prepared from the same natural molecule and have a vapour tension that is less than ethyl, propyl or butyl acetates. This property induces a lesser emission of volatile organic compounds (VOCs).

A ministerial order dated 1 Mar. 1993 considers any compound which, with the exclusion of methane, contains carbon and hydrogen (which may be substituted with other atoms such as halogens, oxygen, sulphur, nitrogen or phosphorus, with the exception of carbon oxides and of carbonates), and which is in the gaseous or vapour state under normal temperature and pressure conditions, to be a volatile organic compound (VOC). European Directive 1999/13/EC completes this definition and adds that any organic product having a vapour pressure of greater than 10 Pa (approximately 0.075 mmHg) is considered to be a VOC. In the United States, the vapour tension threshold selected is much lower, namely 0.13 Pa (approximately 0.001 mmHg) under normal conditions. In Australia, VOCs are defined as chemical compounds having a vapour pressure of greater than 27 Pa at 25° C.

Among VOCs, mention may in particular be made, without this list being exhaustive, of solvents, degreasing agents, dissolving agents, preserving agents, cleaning agents and dispersing agents. The legislations, although variable according to country, all tend towards a reduction in the emission of these VOCs. In France, the regulations have been modified in order to take into account the requirements derived from European Directive 1999/13/EC of 11 Mar. 1999 relating to the reduction of VOC emissions due to the use of organic solvents in certain activities and installations. Consequently, it is essential to find cosmetic compositions which have limited VOC levels.

The molecules presented in Table 1 have a vapour tension that is substantially lower than the vapour tensions of the solvents normally used in nail varnishes, ethyl acetate and butyl acetate. In order to clearly and vigorously evaluate the reduction in VOC emission brought about by the use of novel solvents of natural origin, the study was based on a model published in 2002 by the ADEME [French Environmental and Energy Control Agency) in the report titled “VOC emissions in the paint, varnish, printing ink, glue and adhesive production sector”. The model used, the Clements model, makes it possible to calculate the VOC emissions engendered by surface evaporation during the production of a varnish in an open tank.

The emissions of each solvent present in a varnish composition are calculated by the equation:

$$Ei = \frac{Mi \cdot Ki \cdot A \cdot Pi \cdot 3600 \cdot hr}{R \cdot T} \cdot N$$

where:
Ei is the emission of the species i in kg per year,
Mi is the molecular mass of the species i in g/mol,
Ki is the mass transfer coefficient of the species i in m·s⁻¹, from the liquid phase to the gas phase,
Pi is the vapour pressure of the species i in kPa,
Hr is the duration in hours of the manufacturing operation,
A is the free surface area of the tank in m²,
R is the ideal gas constant (8.314 J·K⁻¹·mol⁻¹),
T is the absolute temperature in Kelvin, and
N is the number of identical manufacturing operations in the year.
The coefficient of transfer of the species i to the gas phase (Ki) can be determined by the equation:

$$Ki = 0.00250 \cdot {V}^{0.78} \cdot {( \frac{18}{Mi} )}^{1 / 3}$$

where:
Ki is expressed in m·s⁻¹,
V is the speed of the air above the tank in m·s⁻¹,

Mi is the molar mass of the species i in g·mol⁻¹.

TABLE 1
Comparison between the vapour tensions and the flashpoints of
the usual solvents for nail varnish (ethyl and butyl acetate)
and of the solvents of natural origin

| | Vapour tension at 25° C. | |
|---|---|---|
| | (Pa) | Flashpoint (° C.) |
| Ethyl acetate | 10300 | −4 |
| Butyl acetate | 1990 | 24 |
| Ethanol | 7827 | 12.8 |
| Isoamyl acetate | 757 | 25 |
| Isoamyl alcohol | 555 | 45.6 |
| Isoamyl carbonate | 374 | 55.5 |
| Ethyl isoamyl ether | 3413 | 9 |
| Methyl isovalerate | 2426 | 19.4 |
| Ethyl isovalerate | 1047 | 26.7 |

The term “fusel oil”, of German origin, translates as “lower alcohol or alcohol of poor quality”. Today, this term denotes all higher alcohols obtained at various stages of fermentation. The compounds of fusel oil can be classified in two major groups (PATIL A. G. S. M. et al. International Sugar Journal, (2002), 104, 51-54, 56-58):

the high boiling fraction (HBF), Bp >132° C. It represents only 1 to 5% of fusel oil. The constituents of this fraction can be classified in three groups (SHORUIGIN, P. Pet al. Ber. (1933). 66B: 1087-1093; SHORUIGIN, P P et al., Zhurnal Obshchei Khimii (1934), 4 372-394):
acidic compounds (10-25%): higher alcohols (hexanol, heptanol, octanol, nonanol), fatty acids (butyric, valeric, caproic, caprylic, pelargonic, capric, lauric, myristic, palmitic) and esters thereof (acetates, butyrates),
basic compounds (5-10%): di-, tri- and tetramethylpyrazines, and
neutral compounds (60-80%): terpenes;

the low boiling fraction (LBF), Bp <132° C. This fraction represents the major portion of fusel oil (95-98%) (PATIL already cited).

Table 2 hereinafter presents some compositions of the LBF fraction of fusel oil.

TABLE 2
Composition of the LBF fraction of fusel oils of various origins (%)

| | | | | | | Iso- | Active | Iso- |
|---|---|---|---|---|---|---|---|---|
| | H₂O | EtOH | PrOH | Iso-PrOH | BuOH | BuOH | AmOH | AmOH |
| Beet molasses | — | 10.0 | 0.6 | — | 2.0 | 0.2 | 3.0 | 73.0 |
| Beet molasses | — | 12.4 | 3.5 | — | — | 9.5 | — | 74.6 |
| (KUCUK Z. et | | | | | | | | |
| al. Turkish | | | | | | | | |
| Journal of | | | | | | | | |
| Chemistry | | | | | | | | |
| (1998) 22(3), | | | | | | | | |
| 289-300) | | | | | | | | |
| Sugarbeet | — | 3.96 | — | | 9.61 | 5.28 | — | 76.86 |
| molasses | | | | | | | | |
| (KHEDR, M. A. | | | | | | | | |
| et al. Pakistan | | | | | | | | |
| Journal of | | | | | | | | |
| Scientific and | | | | | | | | |
| Industrial | | | | | | | | |
| Research (1994) | | | | | | | | |
| 37 (11) 488-490) | | | | | | | | |
| Molasses | — | nd | 13.2 | — | 0.2-0.7 | 15.8 | 18.4 | 37.4 |
| (ULLMAN'S | | | | | | | | |
| 1981) | | | | | | | | |
| Potatoes | — | nd | 14.0 | — | 0.5 | 15.5 | 15.0 | 55.0 |
| (ULLMAN'S | | | | | | | | |
| 1981) | | | | | | | | |
| Fruits | — | nd | 8.0 | — | 2.0 | 19.0 | 14.0 | 57.0 |
| (ULLMAN'S | | | | | | | | |
| 1981) | | | | | | | | |
| Cereals | — | nd | 9.1 | — | 0.2-0.7 | 19.0 | 20.0 | 13.0 |
| (ULLMAN'S | | | | | | | | |
| 1981) | | | | | | | | |
| Waste | — | nd | 7.0 | — | — | 22.0 | 13.0 | 55.0 |
| (ULLMAN'S | | | | | | | | |
| 1981) | | | | | | | | |

The percentages of each alcohol vary greatly according to the starting material used for the fermentation, but also according to the method of fermentation or of distillation.

Fusel oil is a relatively viscous liquid that is straw yellow to dark red in colour and has an unpleasant odour. Before the development of processes of chemical synthesis, fusel oil was the only commercial source of amyl alcohols.

Subsequent to the production of 10001 of alcohol, between 1 and 111 of fusel oil can be obtained. This percentage depends on the starting material used and on the fermentation and distillation conditions, as illustrated in Table 3 below.

TABLE 3
Fusel oil production yields
(according to PATIL A.G.S. already cited)

| Starting material | Fusel oil |
|---|---|
| Sugarbeet molasses | 1-5% |
| Maize | 4-5% |
| Wheat | 2-3% |
| Potatoes | 5-11% |

The uses of fusel oil are quite diverse and have changed a great deal over the years. Before the 1930s, fusel oil was used only as a source of amyl alcohols. Around 1935, several studies begin to refer to the use of fusel oil or of its derivatives as solvents for paints, lacquers and nitrocellulose (MAKINO Z. et al., JP 111027; TING H. W. Research Inst. Ann. Rept. Bur. Chem. (1936), 3, 75; CAVALIE H. R. et al., FR988540). However, the vast majority of authors were studying the distillation and purification of this distillation residue. Koslov et al. (Zhurnal Prikladnoi Khimii, (1954), 27, 223-225) used fusel oil as a flotation agent for copper and zinc ores. Gukasyan et al. (Tsvetnye Metallyst, (1979), 12, 61-62) used fusel oil in place of trioctylamine for extracting rhenium from solutions thereof. Other authors made use of fusel oil phosphates for extracting thallium(III) (SRIVASTAVA T. N. et al. Ladbev Part A: Physical Sciences, (1971), 9, (34), 178-182), titanium(IV) (HASAN S. H. et al., Asian Journal of Chemistry, (1993), 5(2), 266-277) or zirconium(IV) (HASAN S. H. et al., Acta Chimica Hungaria, (1990), 127(2), 235-245).

Amyl alcohols are more liposoluble than propyl and butyl alcohols, and therefore fusel oil quickly found its place as an additive for petroleum products and hydraulic fluids. It was often used in mixtures of fuels for diesel engines for agricultural use (GORMAN J. W., U.S. Pat. No. 4,585,461; ZHANG G. M. CN 1068844; KARAOSMANOGLU F. et al., Energy Sources (1997), 19(6), 567-577).

Fusel oil esters can be used industrially as plasticizers (GHUIBA F. M. et al., Indian Journal of Technology, (1985), 23(8), 309-311), lubricants (OZGULSUN A. et al., Journal of the American Oil Chemists' Society, (2000), 77(1), 105-109, extracting agents and flavourings (WELSCH F. W. et al., Journal of Food Science (1989), 54(6), 1565-1568; YOSHIDA N. JP 01030647; ADNAN A. et al., Pakistan Journal of Scientific and Industrial Research (1994), 37(11), 449-452) or emulsifiers (LOU Y., CN 1053085). Recently, several Russian researchers have studied the use of the acetates derived from starch fusel oil as a solvent for industrial coatings or as a dissolving agent for nail varnishes (RU 2 174 974; KORYSTIN S. I. et al., Tekhnika Mashinostroenyia (2002), 6 98-104; RU 2 194 492). None of these documents discloses the use of a solvent of plant origin as a solvent for preparing nail varnishes.

### DETAILED DESCRIPTION

Now, the inventors have discovered that solvents of plant origin, in particular esterified fusel oil, can be used as a solvent in varnish preparation, and make it possible to obtain varnishes that are easy to apply, for which the drying time is of the order of 2 to 4 minutes and which exhibit good elasticity. The subject of the present invention is therefore a varnish composition for cosmetic or pharmaceutical use, characterized in that it comprises one or more solvent(s) exclusively of plant origin. In an advantageous embodiment of the invention, the solvent is composed of one or more fusel oil derivatives chosen from the group comprising fusel oil acetates (esterified fusel oil), fusel oil carbonates, fusel oil ethers or isovalerates. In the present application, all these compounds will be called “fusel oil derivatives”.

According to the present invention, the fusel oil used is a mixture of C₁-C₅, preferably C₂-C₅, alcohols, containing:
0 to 95%, advantageously 30 to 90%, even more advantageously 50 to 80%, of 3-methyl-1-butanol,
0.5 to 20%, advantageously 5 to 15%, of a mixture of 1-propanol and of 2-propanol, and
2 to 63%, advantageously 10 to 20%, of a mixture of 1-butanol and of 2-methyl-1-propanol;
   or containing:
0 to 100%, advantageously 30 to 90%, even more advantageously 60 to 80%, of 3-methyl-1-butanol,
0 to 50%, advantageously 5 to 20%, of a mixture of 1-butanol and of 2-methyl-1-propanol, and
0 to 20% of a mixture of 1-propanol and of 2-propanol,
0 to 20% of ethanol, and
0 to 20%, advantageously 0 to 5%, even more advantageously less than 1%, of water.

In the context of the invention, it is possible to carry out, using fusel oil, various types of reactions in order to obtain:
acetates by esterification, in particular acetates chosen from the group comprising isoamyl, butyl, isobutyl, propyl, isopropyl and ethyl acetates,
carbonates by transesterification of dimethyl carbonate (DMC), in particular carbonates chosen from the group comprising methyl isoamyl carbonate, methyl butyl carbonate, methyl isobutyl carbonate, methyl propyl carbonate, methyl isopropyl carbonate and methyl ethyl carbonate,
ethers by etherification, in particular ethers chosen from the group comprising isoamyl ethyl ether, isobutyl ethyl ether, butyl ethyl ether, propyl ethyl ether and isopropyl ethyl ether,
isovalerates by esterifying oxidation, in particular methyl or ethyl isovalerates.
   For each of these reactions, the reaction medium obtained after separation of the catalysts can be distilled in order to obtain a single molecule. It can also be used as it is, i.e. as a mixture of several molecules of the same family.

In the case of complete purification, the invention makes it possible to form, from the fusel oil:
isoamyl acetate in the case of an esterification,
methyl isoamyl carbonate in the case of a transesterification,
isoamyl ethyl ether in the case of an etherification,
methyl 3-methylbutanoate or ethyl 3-methylbutanoate, also called methyl or ethyl isovalerates, in the case of an oxidation followed by an esterification, depending on the solvent in which the reaction is carried out: methanol or ethanol.

If a partial distillation, or advantageously no distillation, is carried out after reaction, the invention makes it possible to form, from the fusel oil:
a mixture of acetates in the case of an esterification, the composition of which is:
   0 to 100%, advantageously 30 to 90%, even more advantageously 50 to 80%, of isoamyl acetate,
   0 to 20%, advantageously 5 to 15%, of a mixture of butyl and isobutyl acetates,
   0 to 20%, advantageously 5 to 10%, of a mixture of propyl and isopropyl acetates,
   0 to 20%, advantageously 5 to 15%, of ethyl acetate,
   0 to 20%, advantageously 0 to 5%, even more advantageously less than 1%, of water;
a mixture of carbonates in the case of a transesterification of dimethyl carbonate, the composition of which is:
   0 to 100%, advantageously 30 to 90%, even more advantageously 50 to 80%, of methyl isoamyl carbonate,
   0 to 20%, advantageously 5 to 15%, of a mixture of methyl isobutyl carbonate and of methyl butyl carbonate,
   0 to 20%, advantageously 5 to 10%, of a mixture of methyl propyl carbonate and of methyl isopropyl carbonate,
   0 to 20%, advantageously 5 to 15%, of ethyl carbonate,
   0 to 20%, advantageously 0 to 5%, even more advantageously less than 1%, of water,
   0 to 10%, advantageously 0 to 1%, even more advantageously 0%, of a mixture of high molecular weight carbonates, such as diisoamyl carbonate, diisobutyl carbonate or diisopropyl carbonate;
a mixture of ethers in the case of an etherification, the composition of which is:
   0 to 100%, advantageously 30 to 90%, even more advantageously 50 to 80%, of isoamyl ethyl ether,
   0 to 20%, advantageously 5 to 15%, of a mixture of isobutyl ethyl ether and of butyl ethyl ether,
   0 to 20%, advantageously 5 to 10%, of a mixture of propyl ethyl ether and of isopropyl ethyl ether,
   0 to 20%, advantageously 0 to 5%, even more advantageously less than 1%, of water,
a mixture of esters in the case of an oxidation followed by an esterification, the composition of which is:
   0 to 100%, advantageously 30 to 90%, even more advantageously 50 to 80%, of ethyl or methyl isovalerate,
   0 to 20%, advantageously 5 to 15%, of a mixture of ethyl or methyl butyrate and of ethyl or methyl isobutyrate,
   0 to 20%, advantageously 0 to 5%, of ethyl or methyl propionate,
   0 to 20%, advantageously 0 to 5%, of ethyl or methyl acetate,
   0 to 20%, advantageously 0 to 5%, even more advantageously less than 1%, of water.
      In the context of the present invention, each of the molecules can be used pure, as a mixture with other molecules of the same family, as a mixture with other families of molecules synthesized from fusel oil, or alternatively as a mixture with other solvents of natural origin.

The fusel oil used in the context of the invention can also be used in esterified form and comprising a mixture of C₃-C₇ acetate containing:
0 to 95%, advantageously 30 to 90%, even more advantageously 50 to 80%, of a mixture of secondary amyl acetate, of isoamyl acetate and of n-amyl acetate,
0.5 to 20%, advantageously 5 to 15%, of a mixture of n-propyl acetate and of isopropyl acetate, and
2 to 63%, advantageously 10 to 20%, of a mixture of n-butyl acetate and of isobutyl acetate.
   In the context of the present invention, the fusel oil is obtained from at least one plant chosen from the group comprising sugar cane or beet molasses, potato, cereals, sweet potato, fruits and waste from these plants.

The esterification of the fusel oil can be carried out by any method known to those skilled in the art, in particular (1) by Fischer esterification, reaction between an alcohol and a carboxylic acid in the presence of an inorganic acid, (2) by reaction between an alcohol and a carboxylic acid in the presence of an ion exchange resin, (3) by reaction between an alcohol and an acid anhydride or (4) by reaction between an alcohol and an acid chloride. Advantageously, the esterification is carried out by reaction with acetic acid in the presence of an acid catalyst such as sulphuric acid or hydrochloric acid, or in the presence of an acidic ion exchange resin used batchwise or in a single column.

By way of example of a resin advantageously used according to the invention, mention may be made of the resins Dowex DR-2030, Lewatit® from Bayer, Purolite C T or Amberlyst® from Rohm and Haas, preferably Amberlyst® 15 wet. The carbonates can be prepared by any method known to those skilled in the art, in particular by transesterification of dimethyl carbonate, with an alcohol derived from fusel oil. The ethers can also be prepared by any method known to those skilled in the art, in particular by etherification of an alcohol derived from fusel oil. The isovalerates can be obtained by any method known to those skilled in the art, in particular by oxidation followed by esterification of an alcohol derived from fusel oil.

The varnish compositions according to the invention also comprise at least one polyester resin and a film-forming agent that is soluble in the solvent derived from the fusel oil, said film-forming agent being advantageously nitrocellulose or one of its derivatives, in particular a collodion. In accordance with the invention, the solvent or the mixture of solvents derived from the fusel oil advantageously represents from 55 to 90% by weight of the composition, advantageously from 60 to 80%, even more advantageously from 65 to 75%.

The varnish compositions according to the invention may also comprise one or more adjuvants normally used in the cosmetics or pharmaceutical field, chosen from the group comprising plasticizers, diluents, dyes, organic and inorganic pigments, thixotropic agents, sunscreens of UVA and UVB type, dispersants, wetting agents, matting agents, adhesive agents, coating agents, rheological agents, preserving agents, antioxidants, thickeners, hardening agents and propenetrating agents. The varnishes according to the invention may also comprise at least one active principle for cosmetic or therapeutic use, chosen from the group comprising antifungal agents, corn-removing agents, virucidal agents, vermicidal agents, antibiotics, antibacterial agents, steroidal or non-steroidal anti-inflammatories, antiparasitic agents, antiviral agents and immunosuppressants. The concentrations of active principle are advantageously between 0.001 and 10% by weight relative to the total weight of the varnish.

The varnishes according to the invention are prepared by usual methods in the field. The varnishes according to the invention can be used as a coating for nails, in particular as a nail varnish for cosmetic use or as a nail varnish for pharmaceutical use, in particular in the treatment of the following dermatological diseases: onychomycosis, chloronychia, paronychias, erysipeloid, onychorrhexis, gonorrhoea, swimming-pool granuloma, larva migrans, leprosy, Orf nodule, milkers' nodules, herpetic whitlow, acute bacterial perionyxis, chronic perionyxis, sporotrichosis, syphilis, tuberculosis verrucosa cutis, tularemia, tungiasis, peri- and subungual warts, zona, dermatological diseases with an effect on the nails, such as psoriasis, pustular psoriasis, alopecia aerata, parakeratosis pustulosa, contact dermatosis, Reiter's syndrome, parakeratosis pustulosa, psoriasiform acral dermatitis, lichen planus, idiopathic atrophy in the nails, lichen nitidus, lichen striatus, inflammatory linear verrucous epidermal naevus (ILVEN), alopecia, pemphigus, bullous pemphigoid, acquired epidermolysis bullosa, Darier's disease, pityriasis rubra pilaris, palmoplantar keratoderma, contact eczema, polymorphic erythema, scabies, Bazex syndrome, systemic scleroderma, systemic lupus erythematosus, chronic lupus erythematosus and dermatomyositus. The subject of the present invention is also the use of one or more solvent(s) of plant origin, derived from fusel oil, in a varnish composition for cosmetic or pharmaceutical use.

The examples that follow illustrate the invention.

Example 1 illustrates the composition of the various fusel oils according to their origin.

Examples 2 to 4 illustrate the preparation of various families of molecules from fusel oil.

Example 5 illustrates a reference varnish formulation in which the solvents are ethyl acetate and butyl acetate.

Examples 6 to 11 illustrate varnish formulations in which the solvent(s) is (are) exclusively of natural origin and derived from fusel oil.

The various varnishes are prepared by techniques conventionally used in the field.

### EXAMPLE 1

### Identification of the Fusel Oil Components

The compositions of three fusel oils were determined by gas chromatography according to the techniques known to those skilled in the art, and are given in Table 4 below.

TABLE 4
Fusel oil compositions (% by weight)

| | Maize fusel oil | Beet fusel oil | Wheat fusel oil |
|---|---|---|---|
| EtOH | 1.69 | 11.32 | 17.65 |
| PrOH | 0.03 | 0.08 | 0.09 |
| iso-PrOH | — | 0.05 | — |
| BuOH | 0.14 | 0.06 | 0.15 |
| iso-BuOH | 3.43 | 0.25 | 0.69 |
| 2-BuGH | — | — | — |
| iso-AmOH | 74.84 | 75.73 | 57.62 |
| Total* (%) | 80.13 | 87.49 | 76.2 |

*The remainder of the fusel oil being essentially water.

### EXAMPLE 2

### Synthesis of Isoamyl Acetate by Esterification of the Fusel Oil

The fusel oil is esterified with acetic acid (stoichiometric amounts), in the presence of a highly acidic ion exchange resin Amberlyst® 15 (2% relative to the total mass). The mixture is heated at 70° C. for 2 h so as to form a mixture of acetates. The residue is filtered so as to be regenerated and the reaction medium is distilled so as to obtain the isoamyl acetate (Bp: 131° C.) in the form of a colourless liquid with a fruity smell.

### EXAMPLE 3

### Synthesis of Isoamyl Carbonate by Transesterification of Dimethyl Carbonate with Fusel Oil

The synthesis of isoamyl methyl carbonate is carried out with an excess of dimethyl carbonate (DMC). The fusel oil is added to 5 equivalents of DMC and the reaction is catalyzed by potassium carbonate K₂CO₃. Heating of the reaction medium at 100° C. for 1 h results in a 75% conversion rate of the fusel oil with a 95% selectivity for methyl isoamyl carbonate. The mixture is filtered so as to recover the catalyst and then purified by distillation so as to obtain the desired fraction. Total purification produces the isoamyl methyl carbonate (Bp: 151-152° C.) in the form of a colourless liquid with a fruity smell.

### EXAMPLE 4

### Synthesis of Isoamyl Ethyl Ether by Etherification

Isoamyl ethyl ether is formed by nucleophilic substitution of the isoamyl alcohol contained in the fusel oil onto iodoethane, according to a Williamson reaction. The synthesis is carried out without solvent, at ambient temperature, by mixing isoamyl alcohol, iodoethane and potassium hydroxide in stoichiometric proportions. A catalytic amount of polyethylene glycol (PEG 300) is used as phase transfer catalyst. After stirring for 4 h, all the iodoethane has been consumed and the isoamyl alcohol conversion rate reaches 94%. Filtration of the potassium iodide formed and then distillation at atmospheric pressure makes it possible to obtain the isoamyl ethyl ether (Bp: 103-104° C.) in the form of a colourless liquid with a fruity smell.

### EXAMPLE 5

### Colourless Nail Varnish Formulation (Reference Varnish)

| Function | Reference composition (colourless 056) | |
|---|---|---|
| Solvents | Ethyl acetate | 51% |
| | Butyl acetate | 21.2% |
| Film-forming | Nitrocellulose E27 (30% IPA*) | 12.8% |
| agent | | |
| Plasticizer | Acetyl tributyl citrate | 6% |
| Resin | Polyester resin | 9% |
| Total | | 100% |

*TPA = isopropyl alcohol

### EXAMPLE 6

### Colourless Nail Varnish Formulation

| Function | Novel solvent composition | |
|---|---|---|
| Solvents | Fusel acetate (mixture of C₂-C₅ acetates) | 72.2% |
| Film-forming | Nitrocellulose E27 (30% IPA) | 12.8% |
| agent | | |
| Plasticizer | Acetyl tributyl citrate | 6% |
| Resin | Polyester resin | 9% |
| Total | | 100% |

### EXAMPLE 7

### Colourless Nail Varnish Formulation

| Function | Novel solvent composition | |
|---|---|---|
| Solvents | Pure isoamyl acetate | 72.2% |
| Film-forming | Nitrocellulose E27 (30% IPA) | 12.8% |
| agent | | |
| Plasticizer | Acetyl tributyl citrate | 6% |
| Resin | Polyester resin | 9% |
| Total | | 100% |

### EXAMPLE 8

### Colorless Nail Varnish Formulation

| Function | Novel solvent composition | |
|---|---|---|
| Solvents | Isoamyl acetate | 35.6% |
| | Ethanol | 35.6% |
| Film-forming | Nitrocellulose E27 (30% IPA) | 12.8% |
| agent | | |
| Plasticizer | Acetyl tributyl citrate | 6% |
| Resin | Polyester resin | 9% |
| Total | | 100% |

### EXAMPLE 9

### Colourless Nail Varnish Formulation

| Function | Novel solvent composition | |
|---|---|---|
| Solvents | Isoamyl carbonate | 31% |
| | Ethanol | 31% |
| Film-forming | Nitrocellulose E27 (30% IPA) | 19% |
| agent | | |
| Plasticizer | Acetyl tributyl citrate | 6% |
| Resin | Polyester resin | 13% |
| Total | | 100% |

### EXAMPLE 10

### Colourless Nail Varnish Formulation

| Function | Novel solvent composition | |
|---|---|---|
| Solvents | Isoamyl acetate | 31% |
| | Methyl isoamyl ether | 31% |
| Film-forming | Nitrocellulose E15 (30% IPA) | 19% |
| agent | | |
| Plasticizer | Acetyl tributyl citrate | 6% |
| Resin | Polyester resin | 13% |
| Total | | 100% |

### EXAMPLE 11

### Colourless Nail Varnish Formulation

| Function | Novel solvent composition | |
|---|---|---|
| Solvents | Methyl isovalerate | 62% |
| Film-forming | Nitrocellulose E15 (30% IPA) | 19% |
| agent | | |
| Plasticizer | Acetyl tributyl citrate | 6% |
| Resin | Polyester resin | 13% |
| Total | | 100% |

Evaluation of the Ease of Application of the Films:

The reference 056 and the formulations according to the invention were applied, using a Touzart-Matignon applicator, onto contrast cards, as wet films 150 μm thick.

Drying Time:

The measurements of the drying time of the formulations mentioned in the examples were carried out with a drying time device, on a film of 100 μm, at a constant temperature of 20° C. All the films exhibit a drying time similar to that of the reference time, which is 3 minutes.

Evaluation of the VOC Emissions:

The parameters taken into account for calculating the VOC emissions are as follows:

exposure time, i.e. the time necessary to carry out the mixing of the various components of the varnish: 3 h,
number of formulations produced in 1 year: 300,
mean ambient temperature: 20° C.,
speed of the air above the tank: 0.05 m/s,

tank diameter: 1.80 m, which corresponds to a 500 l tank.

| | | Reduction in |
|---|---|---|
| | VOC emission | emissions relative |
| Formulation | (kg/year) | to the reference |
| Example 5 (reference varnish) | 1262.8 | 0% |
| ethyl acetate | | |
| butyl acetate | | |
| Example 6 | 406.9 | −68% |
| fusel acetates | | |
| (mixture of C₂-C₅ acetates) | | |
| Example 7 | 163 | −87% |
| isoamyl acetate | | |
| Example 8 | 509 | −60% |
| isoamyl acetate | | |
| ethanol | | |
| Example 9 | 513.5 | −59% |
| isoamyl carbonate | | |
| ethanol | | |
| Example 10 | 380.7 | −70% |
| isoamyl acetate | | |
| isoamyl ethyl ether | | |
| Example 11 | 407.4 | −68% |
| methyl isovalerate | | |

All the formulations according to the invention exhibit a VOC emission reduced by at least 50% relative to the reference formulation.

The invention claimed is:

1. A varnish composition for cosmetic or pharmaceutical use, comprising at least one solvent exclusively of plant origin.

2. The varnish composition according to claim 1, wherein the solvent exclusively of plant origin is comprised of at least one fusel oil derivative.

3. The varnish composition according to claim 2, wherein the at least one fusel oil derivative is chosen from the group comprising: acetates, carbonates, ethers and isovalerates derived from fusel oil.

4. The varnish composition according to claim 3, wherein the acetates are chosen from the group comprising: isoamyl, butyl, isobutyl, propyl, isopropyl and ethyl acetates.

5. The varnish composition according to claim 3, wherein the carbonates are chosen from the group comprising: methyl isoamyl carbonate, methyl butyl carbonate, methyl isobutyl carbonate, methyl propyl carbonate, methyl isopropyl carbonate and methyl ethyl carbonate.

6. The varnish composition according to claim 3, wherein the ethers are chosen from the group comprising: isoamyl ethyl ether, isobutyl ethyl ether, butyl ethyl ether, propyl ethyl ether and isopropyl ethyl ether.

7. The varnish composition according to claim 3, wherein the isovalerates are chosen from the group comprising: ethyl isovalerate and methyl isovalerate.

8. The varnish composition according to claim 3, wherein the solvent comprises a fusel oil which is a mixture of C₁-C₅, preferably C₂-C₅, alcohols, containing:
0 to 95%, advantageously 30 to 90%, even more advantageously 50 to 80%, of 3-methyl-1-butanol,
0.5 to 20%, advantageously 5 to 15%, of a mixture of 1-propanol and of 2-propanol, and
2 to 63%, advantageously 10 to 20%, of a mixture of 1-butanol and of 2-methyl-1-propanol.

9. The varnish composition according to claim 1, wherein the solvent comprises a fusel oil which is a mixture of C₁-C₅, preferably C₂-C₅, alcohols, containing:
0 to 100%, advantageously 30 to 90%, even more advantageously 60 to 80%, of 3-methyl-1-butanol,
0 to 50%, advantageously 5 to 20%, of a mixture of 1-butanol and of 2-methyl-1-propanol,
0 to 20% of a mixture of 1-propanol and of 2-propanol,
0 to 20% of ethanol, and
0 to 20%, advantageously 0 to 5%, even more advantageously less than 1%, of water.

10. The varnish composition according to claim 3, wherein the fusel oil derivative used is a mixture of acetates, obtained by esterification of the fusel oil, the composition of which is:
0 to 100%, advantageously 30 to 90%, even more advantageously 50 to 80%, of isoamyl acetate,
0 to 20%, advantageously 5 to 15%, of a mixture of butyl and isobutyl acetates,
0 to 20%, advantageously 5 to 10%, of a mixture of propyl and isopropyl acetates,
0 to 20%, advantageously 5 to 15%, of ethyl acetate, and
0 to 20%, advantageously 0 to 5%, even more advantageously less than 1%, of water.

11. The varnish composition according to claim 3, wherein the fusel oil derivative used is a mixture of carbonates, obtained by transesterification of dimethyl carbonate, the composition of which is:
0 to 100%, advantageously 30 to 90%, even more advantageously 50 to 80%, of methyl isoamyl carbonate,
0 to 20%, advantageously 5 to 15%, of a mixture of methyl isobutyl carbonate and of methyl butyl carbonate,
0 to 20%, advantageously 5 to 10%, of a mixture of methyl propyl carbonate and of methyl isopropyl carbonate,
0 to 20%, advantageously 5 to 15%, of ethyl carbonate,
0 to 20%, advantageously 0 to 5%, even more advantageously less than 1%, of water, and
0 to 10%, advantageously 0 to 1%, even more advantageously 0%, of a mixture of high molecular weight carbonates, such as diisoamyl carbonate, diisobutyl carbonate or diisopropyl carbonate.

12. The varnish composition according to claim 3, wherein the fusel oil derivative used is a mixture of ethers, obtained by etherification of fusel oil, the composition of which is:
0 to 100%, advantageously 30 to 90%, even more advantageously 50 to 80%, of isoamyl ethyl ether,
0 to 20%, advantageously 5 to 15%, of a mixture of isobutyl ethyl ether and of butyl ethyl ether,
0 to 20%, advantageously 5 to 10%, of a mixture of propyl ethyl ether and of isopropyl ethyl ether, and
0 to 20%, advantageously 0 to 5%, even more advantageously less than 1%, of water.

13. The varnish composition according to claim 3, wherein the fusel oil derivative used is a mixture of esters, obtained by oxidation and then esterification of fusel oil, the composition of which is:
0 to 100%, advantageously 30 to 90%, even more advantageously 50 to 80%, of ethyl or methyl isovalerate,
0 to 20%, advantageously 5 to 15%, of a mixture of ethyl or methyl butyrate and of ethyl or methyl isobutyrate,
0 to 20%, advantageously 0 to 5%, of ethyl or methyl propionate,
0 to 20%, advantageously 0 to 5%, of ethyl or methyl acetate, and
0 to 20%, advantageously 0 to 5%, even more advantageously less than 1%, of water.

14. The varnish composition according to claim 1, wherein the solvent comprises a fusel oil which is obtained from at least one plant chosen from the group comprising: sugar cane or beet molasses, potato, cereals, sweet potato, fruits and the waste from these plants.

15. The varnish composition according to claim 1, further comprising at least one film-forming polymer soluble in the solvent derived from fusel oil, advantageously nitrocellulose or one of its derivatives, in particular a collodion, and optionally at least one polyester resin.

16. The varnish composition according to claim 1, wherein the solvent or the mixture of solvents represents from 55 to 90% by weight of the composition, advantageously from 60 to 80%, even more advantageously from 65 to 75%.

17. The varnish composition according to claim 1, further comprising one or more adjuvants chosen from the group comprising: plasticizers, diluents, dyes, organic and inorganic pigments, thixotropic agents, sunscreens of UVA and UVB type, dispersants, wetting agents, matting agents, adhesive agents, coating agents, rheological agents, preserving agents, antioxidants, thickeners, hardening agents and propenetrating agents.

18. The varnish composition according to claim 1, further comprising at least one compound chosen from the group comprising: antifungal agents, corn-removing agents, vermicidal agents, virucidal agents, antibiotics, antibacterial agents, anti-inflammatories, steroidal or non-steroidal agents, antiparasitic agents, antiviral agents and immunosuppressants.

19. The varnish composition according to claim 1, wherein the composition is a nail varnish for cosmetic use or for pharmaceutical use.

20. A solvent of plant origin, comprising fusel oil derivatives as a solvent in a cosmetic or pharmaceutical varnish composition.
